(19) United States
(12) Patent Application Publication
(10) Pub. No.: US 2013/0296836 A1
(43) Pub. Date: Nov. 7, 2013

(54) SYSTEM AND METHOD OF PRE-AORTIC GANGLION ABLATION

(71) Applicant: ENIGMA MEDICAL, INC., San Diego, CA (US)

(72) Inventors: Denise Barbut, New York, NY (US); Allan Rozenberg, San Diego, CA (US); Axel Heinemann, New York, NY (US)

(73) Assignee: ENIGMA MEDICAL, INC., San Diego, CA (US)

(21) Appl. No.: 13/787,325

(22) Filed: Mar. 6, 2013

Related U.S. Application Data
(60) Provisional application No. 61/641,599, filed on May 2, 2012.
(60) Provisional application No. 61/724,086, filed on Nov. 8, 2012.
(60) Provisional application No. 61/733,034, filed on Dec. 4, 2012.
(60) Provisional application No. 61/739,396, filed on Dec. 19, 2012.

(51) Int. Cl. A61B 18/14

(52) U.S. Cl. CPC A61B 18/14
USPC 606/14; 606/41; 606/33; 606/20

(57) ABSTRACT

A method of modulating a physiological parameter of a patient is provided. The method includes disabling one or more pre-aortic ganglion cells within a pre-aortic ganglion and improving the physiological parameter. The method further includes destroying a pre-aortic ganglion cell to prevent regeneration.

This application claims priority to U.S. Ser. No. 61/641,599, filed on May 2, 2012, and U.S. Ser. No. 61/724,086, filed on Nov. 8, 2012, and U.S. Ser. No. 61/733,034, filed on Dec. 4, 2012, and U.S. Ser. No. 61/739,396, filed on Dec. 19, 2012, the entireties of which are incorporated herein by reference.

### FIELD OF THE INVENTION

The present invention relates generally to the field of hypertension. More specifically, the present invention relates to a system and method of pre-aortic ganglion ablation for the treatment of hypertension.

### BACKGROUND OF THE INVENTION

Hypertension affects tens of millions of individuals. Untreated hypertension is associated with stroke, heart failure and renal failure. Most patients with hypertension are currently treated pharmacologically, many with multiple medications. A quarter of these patients are resistant to medication and their blood pressure poorly controlled, putting them at added risk for complications.

Activation of the sympathetic nervous system is thought to play a significant role in exacerbating hypertension in the later stages of the disease. Reducing such sympathetic activation has been shown to reduce blood pressure in these circumstances.

Recently, mechanical ablation of the renal nerves surrounding the renal artery has been shown to reduce blood pressure in patients with resistant hypertension. The technique consists of an endovascular, arterial procedure and involves ablation of post-ganglionic sympathetic nerve fibres, accessed through the wall of the renal arteries bilaterally. Renal artery denervation, as the procedure is known, has been shown to reduce systolic and diastolic pressures by up to 20 mm and 10 mm respectively, and to be persistent out to a year following the procedure. The incidence and severity of procedure related and late complications are as yet unknown, as is the long term benefit of blood pressure. Renal nerve fibres regenerate and the hypotensive effect of this ablative procedure may diminish over time.

Therefore, alternatives to these therapies are needed, which provide more significant reductions in blood pressure, persist indefinitely and which are safer, simpler, and less time-consuming.

### BRIEF SUMMARY OF THE INVENTION

The system and method of pre-aortic ganglion cell ablation offers a new effective method of controlling blood pressure in patients with medication resistant hypertension. It also overcomes the shortcomings of renal artery denervation. The present invention provides a system and method for ablating cell bodies within the pre-aortic ganglia for the treatment of hypertension. These ganglionic cells can be accessed endovascularly through the aorta itself, or through the celiac or superior mesenteric arteries. These methods of treating hypertension have not been previously described.

In one aspect of the invention a method of modulating a physiological parameter of a patient is provided, the method including disabling one or more pre-aortic ganglion cells within a pre-aortic ganglion and improving said physiological parameter.

In a further aspect of the invention, a method of modulating a physiological parameter of a patient is provided the method including destroying a pre-aortic ganglion cell to prevent regeneration.

In a further aspect of the invention, a method of modulating a physiological parameter of a patient is provided, the method including denervating one or more cells within a pre-aortic ganglion and improving said physiological parameter such that vessel spasm and dissection are avoided.

In a further aspect of the invention, a method of modulating a physiological parameter of a patient is provided, the method including denervating one or more cells within a pre-aortic ganglion and improving said physiological parameter such that deterioration of renal function is avoided.

### BRIEF DESCRIPTION OF THE DRAWINGS

For a better understanding of the invention, and to show how the same may be carried into effect, reference will now be made, by way of example, to the accompanying drawings, in which:

FIG. 1 depicts Star-shaped meshwork of sympathetic cell bodies within the pre-aortic ganglia, positioned antero-lateral to the aortic wall and closely adherent to it.

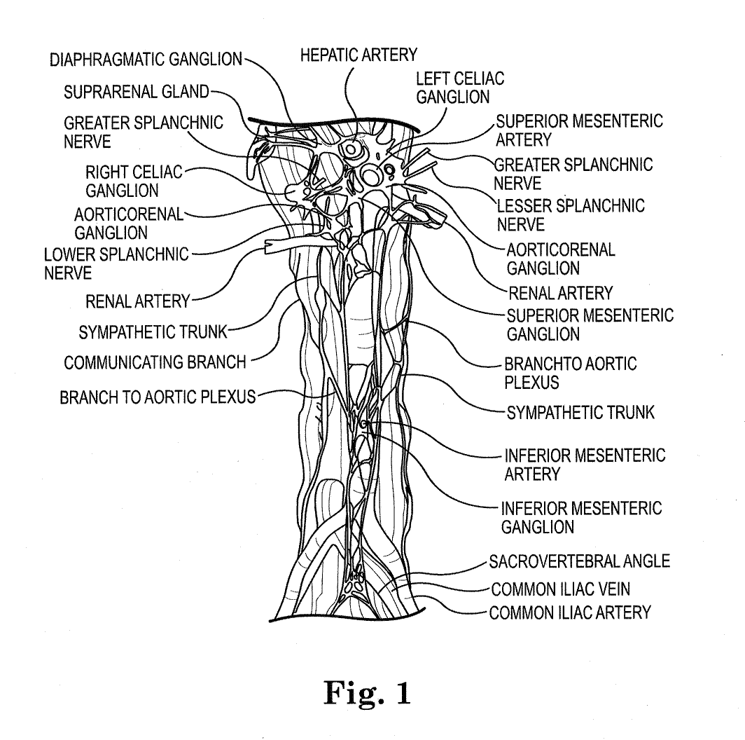

FIG. 2 is a three-dimensional reconstruction of a human aorta showing the position of the celiac and superior mesenteric arteries.

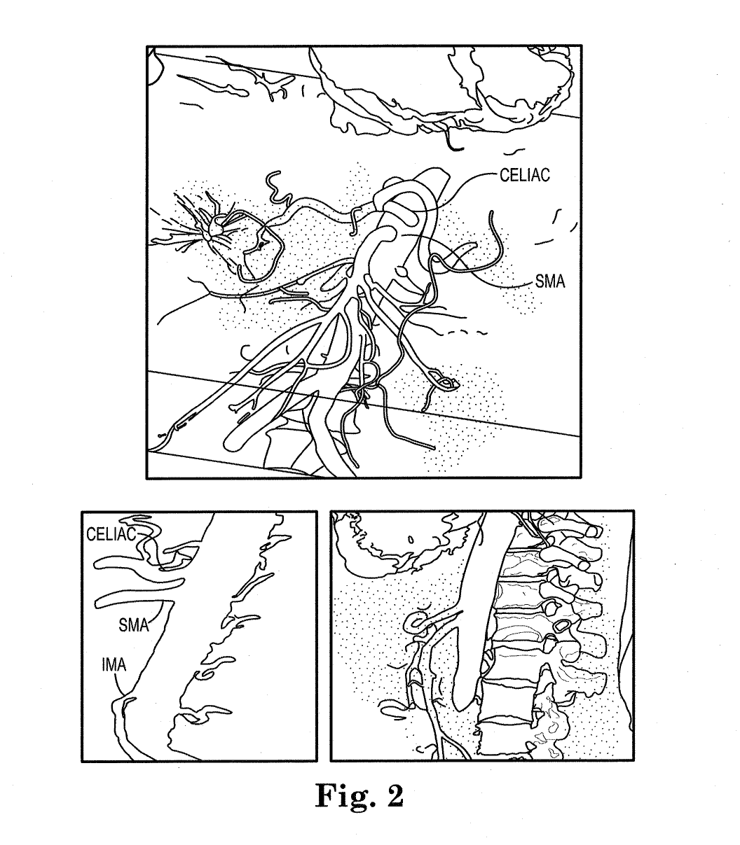

FIG. 3 is an illustration showing the relationship between the right and left pre-aortic ganglia and the aorta.

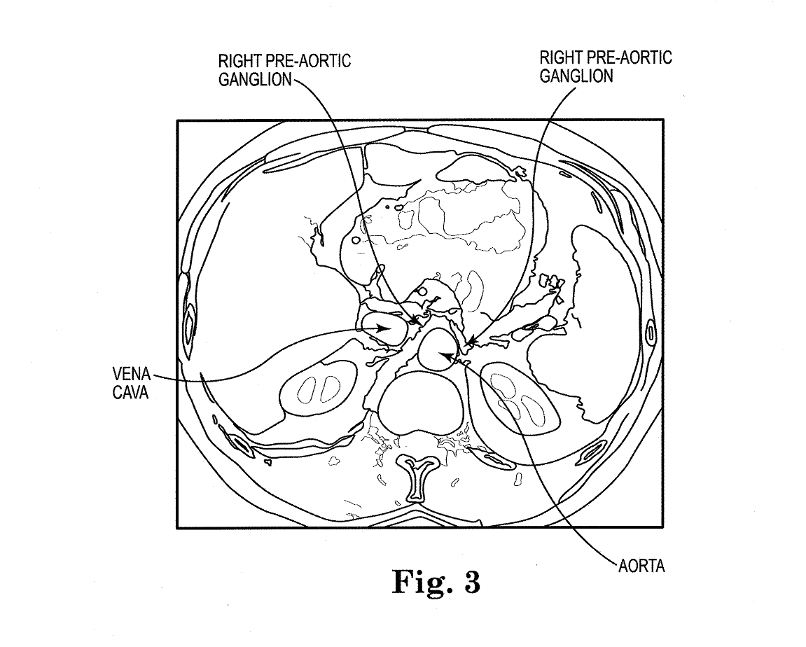

### DETAILED DESCRIPTION OF THE INVENTION

The present invention covers a system and method of ablating a portion of the cell bodies within the pre-aortic ganglia for the treatment of hypertension. These cells can be accessed endovascularly through the aorta itself, or through the celiac or superior mesenteric arteries. The systems and methods of treating hypertension in accordance with the invention have not been previously described.

Afferent sensory nerve fibers from the kidney, the adrenal and the renal artery itself enter the spinal cord through the dorsal root ganglion. They ascend in the spinal cord to the plethora of autonomic control centers within the brain and brainstem. Efferent sympathetic fibers destined for the kidney and adrenal glands, descend in the lateral column of the spinal cord and exit through the ventral root at each spinal level bilaterally. They traverse the white ramus communicantis and the sympathetic paraspinal ganglia in the lower thoracic and upper lumbar spine, and communicate with neighboring paraspinal sympathetic ganglia before they leave the paraspinal space. Pre-ganglionic segmental nerves from T6 to L1, mostly from T8 to T11, then circumnavigate the aorta, terminating at ganglionic cell bodies within the pre-aortic ganglia, namely the splanchnic, mesenteric, celiac, aortico-renal and suprarenal ganglia. Post-ganglionic fibres then track the vasculature, ultimately reaching the renal and adrenal arteries.

Surgical sympathetic denervation for the treatment of resistant hypertension was routinely performed in the 1940's. Such procedures involved removing various combinations of stellate ganglia in the neck, thoraco-lumbar paraspinal sympathetic ganglia, as well as stripping the aortic ganglia. Blood pressure decreases were very significant, and frequently associated with marked postural hypotension. Such surgical procedures were also associated with significant procedural morbidity and mortality, and were rapidly abandoned in favor of pharmacologic treatments which became available in the 1950's. Beta-blockers were followed by ACE inhibitors and other classes of anti-hypertensive medications. Pharmacotherapy became the mainstay of management for hypertensive patients during the second half of the last century. Many patients required more than one medication for adequate control of pressure, and up to a quarter of all remained hypertensive on multiple medications (resistant hypertension).

Recently mechanical means of controlling blood pressure have been revisited, specifically for patients with resistant hypertension. Renal artery denervation (RAD) involves ablating renal nerve fibres surrounding renal arteries bilaterally. The procedure involves advancing a catheter endovascularly into each of the renal arteries, and applying ablative energy through the wall of the artery to destroy some of the renal nerve fibres. The treatment lasts about 40 minutes. Procedure related complications are not uncommon. They include embolization from atheromatous renal arteries to kidneys whose function may already be impaired by chronic hypertension, and renal artery spasm or dissection which may also cause deterioration in renal function. As for efficacy, the procedure is moderately effective. While both systolic and diastolic pressures improve following this treatment, the longer term effect on blood pressure is as yet unknown. Peripheral nerve fibres such as those within the renal nerve typically regenerate, usually at the rate of 1 mm/month. Such regeneration following radiofrequency ablation has been frequently demonstrated (quote abstract). After a significant portion of ablated fibres regenerate, the beneficial effect of the procedure on blood pressure may be lost.

In this invention, we teach that denervation of cell bodies rather than nerve fibres may resolve the shortcomings of renal nerve denervation, and furthermore simplify the procedure itself while reducing complications attributable to the procedure. Regenerative capacity of ganglionic cell bodies is far less than that of nerve fibres. Thus following an ablative procedure, destroyed cell bodies will not recover from the insult and are replaced by glial tissue. Any reduction of blood pressure attributable to the procedure is thus likely to be permanent.

One method of denervating these cell bodies in accordance with the invention includes positioning an ablation device within an aorta of a patient, and advancing it to the level of the superior mesenteric artery or celiac artery, several centimeters above the take-off of the renal arteries. The ganglia are adherent to the antero-lateral aspects of the aorta and lie roughly 0.6 cm below the take-off of the celiac artery on the right and 0.9 cm below the same structure on the left. They can be up to 2.5 cm in length, and are organized somatotopically. These cell bodies are closely adherent to the antero-lateral aortic wall. In an alternative method, the ablation catheter could also be placed within the superior or inferior mesenteric arteries or celiac arteries rather than in the aorta itself. The relevant arteries could be localized angiographically, by ultrasound or by CT/MRI.

The ablation itself could be performed chemically, using pharmacologic agents or heat or cold, by using electric energy or electromagnetic energy such as radiofrequency or ultrasound, including high frequency focused ultrasound and low frequency ultrasound or any other technique that would destroy or partially destroy these structures for the treatment of hypertension. Several parameters may be used to determine further the exact localization of the pre-aortic sympathetic ganglia. By way of example, an energy delivery device may be provided to electrically stimulate the preganglionic fiber endings at the level of the ganglia might be associated with intercostal muscle twitching or contraction or be associated with pain or flushing in the relevant dermatome. Those of skill in the art will appreciate that other similar modes of stimulation may be used and that the energy delivery device may be configured to stimulate or ablate tissue. Lastly, changes in arterial pressure may occur. After the ganglia are localized, the mode may be switched from electrical stimulation to radiofrequency ultrasonic ablation and other modes known to those of skill in the art. Initially, this might cause blood pressure to increase or decrease abruptly. To prevent significant and sudden changes during the procedure and to be able to continuously monitor blood pressure, a pressure sensor may be added to the energy delivery device. The pressure sensor may be configured to feed information back to the energy delivery device and switch it off if blood pressure increases or decreases more than a predetermined amount.

The most significant benefit of this procedure, as already stated, is the extent and permanence of the hypotension achieved because ganglion cell bodies do not regenerate whereas nerve fibres do. In addition, the inventors have found that this method of treating hypertension is safer, simpler and less time-consuming than endovascular renal nerve ablation. The aorta is a huge structure easy to access, whereas renal arteries are smaller and not uncommonly stenosed in this population. Furthermore, dislodgement of atheromatous material from the wall of the aorta is unlikely to embolize to the kidneys and more likely to embolize to the lower limbs, sparing the kidneys. Vasospasm and renal artery dissection are not an issue with procedures being performed in the aorta, while they are very common during instrumentation of the renal artery. Furthermore, while renal nerve denervation involves treating both renal arteries, accessing the pre-aortic ganglia consists of a single procedure.

Although the present invention has been described with reference to preferred embodiments, workers skilled in the art will recognize that changes may be made in form and detail without departing from the spirit and scope of the invention.

We claim:

1. A method of modulating a physiological parameter of a patient, comprising disabling one or more pre-aortic ganglion cells within a pre-aortic ganglion and improving said physiological parameter.

2. The method of claim 1 wherein said disabling comprises irreversibly disabling said one or more cells.

3. The method of claim 1 wherein improving said physiologic parameter comprises permanently improving said physiological parameter.

4. A method of modulating a physiological parameter of a patient, comprising destroying a pre-aortic ganglion cell to prevent regeneration.

5. The method of claim 4 wherein said physiological parameter is permanently improved.

6. The method of claims 1 or 4 wherein the physiological parameter is associated with heart failure, hypertension, acute myocardial infarction, renal disease, chronic renal failure, obesity, diabetes, ischemic bowel syndrome, obstructive sleep apnea, disorders of intestinal motility, or peripheral vascular disease.

7. The method of claim 1 further comprising denervating only a portion of the pre-aortic ganglion including cells that innervate a kidney or an adrenal gland.

8. The method of claim 1 wherein disabling said one or more pre-aortic ganglion cells comprises applying an ablative electrical field to said pre-aortic ganglia.

9. The method of claim 1 further comprising stimulating said pre-aortic ganglion; monitoring a physiologic response related to said physiological parameter; applying an ablative energy to said one or more pre-aortic ganglion cells; and improving said physiological parameter.

10. The method of claim 9, wherein the physiologic response includes a change in blood pressure.

11. The method of claim 1 wherein said pre-aortic ganglion is selected from a celiac ganglion, mesenteric ganglion, suprarenal ganglion, inter-mesenteric ganglion, aortico-renal ganglion, and combinations of the foregoing.

12. The method of claim 1 further comprising providing an energy delivery device; positioning said energy delivery device within a vessel proximate the pre-aortic ganglion; and delivering energy through a wall of said vessel.

13. The method of claim 12 wherein positioning the energy delivery device within a vessel proximate the pre-aortic ganglion comprises positioning the energy delivery device within an aorta, a mesenteric artery, or a celiac artery to deliver said energy to said pre-aortic ganglion.

14. The method of claim 13 wherein positioning the energy delivery device proximate the pre-aortic ganglion comprises positioning the device within the aorta between the origin of the superior mesenteric and celiac arteries.

15. The method of claim 9 further comprising stimulating the pre-aortic ganglion with an energy delivery device; and monitoring a blood pressure of the patient.

16. The method of claim 15 wherein monitoring said blood pressure includes monitoring a change in said blood pressure.

17. The method of claim 12 wherein delivering energy comprises delivering any wavelength from the electromagnetic spectrum, including radiofrequency, microwave, ultrasound, high intensity focused ultrasound, low intensity focused ultrasound, infrared waves, electrical energy, laser energy, other sources of thermal energy, and combinations of the foregoing.

18. The method of claim 17 wherein said thermal energy comprises cooling.

19. The method of claim 12 wherein a pressure sensor is placed on the energy delivery device.

20. The method of claim 19 further comprising recording the pressure; transmitting said pressure back to the energy delivery device; stopping the ablation if blood pressure increases or decreases within a predetermined parameter.

21. The method of claim 12 wherein said energy delivery device comprises an expandable framework structure including one or more electrodes thereon.

22. The method of claim 21 wherein said framework structure is cylindrical or spherical.

23. The method of claim 12 wherein said energy delivery device comprises an elongate steerable body including an electrode thereon.

24. The method of claim 12 wherein said energy delivery device comprises a focused ultrasound device.

25. A method of modulating a physiological parameter of a patient, comprising denervating one or more cells within a pre-aortic ganglion and improving said physiological parameter wherein vessel spasm and dissection are avoided.

26. A method of modulating a physiological parameter of a patient, comprising denervating one or more cells within a pre-aortic ganglion and improving said physiological parameter wherein deterioration of renal function is avoided.

27. A method of modulating a physiological parameter of a patient, comprising denervating one or more cells within a pre-aortic ganglion and improving said physiological parameter wherein embolization from a renal artery is avoided.
